(19) United States
(12) Patent Application Publication
(10) Pub. No.: US 2025/0338878 A1
(43) Pub. Date: Nov. 6, 2025

(54) READY TO DRINK ELECTROLYTE SOLUTION

(71) Applicant: KENVUE BRANDS LLC, Summit, NJ (US)

(72) Inventors: Tarique Ali MEER, Mumbia City (IN); Colin PINTO, Ascent (SG)

(21) Appl. No.: 19/273,385

(22) Filed: Jul. 18, 2025

(30) Foreign Application Priority Data
Jan. 20, 2023 (IN) 202311004093

(51) Int. Cl. A23L 2/38; A23L 2/60; A23L 33/125; A23L 33/15; A23L 33/16; A61K 9/00; A61K 31/4415; A61K 31/592; A61K 31/593; A61K 31/714; A61K 33/30; A61K 47/26

(52) U.S. Cl. CPC A23L 2/38; A23L 2/60; A23L 33/125; A23L 33/15; A23L 33/16; A61K 9/0095; A61K 31/4415; A61K 31/592; A61K 31/593; A61K 31/714; A61K 33/30; A61K 47/26

(57) ABSTRACT

Described is a ready to drink electrolyte solution comprising: Sodium from 40 to 120 mg/100 ml, Potassium from 50 to 100 mg/100 ml, Chloride from 55 to 90 mg/100 ml, and Dextrose from 200 to 600 mg/100 ml, wherein an osmolality of the ready to drink electrolyte solution is lower than or equal to 268 mOsm/kg.

### FIELD

The invention generally relates to a drinkable solution, in particular a ready to drink electrolyte solution. For example, the invention relates to a ready to drink electrolyte solution comprising Sodium, Potassium, Chloride, Dextrose and having an osmolality lower or equal to 268 mOsm/kg. The invention also relates to a method of manufacture of said ready to drink electrolyte solution and to the use of said ready to drink electrolyte solution for the rehydration of a human.

### BACKGROUND

Oral Rehydration Salts (ORS) are well known, they are a balanced glucose-electrolyte mixture containing 90 mEq/l of sodium with a total osmolarity of 311 mOsm/l (Oral Rehydration Salts, Production of the new ORS, World Health Organization (WHO), 2006, WHO/FCH/CAH/06.1). One approach has consisted in reducing the osmolarity of ORS solution to avoid possible adverse effects of hypertonicity on net fluid absorption.

The reduced osmolarity ORS containing 75 mEq/l sodium, 75 mmol/l glucose (total osmolarity of 245 mOsm/1) is as effective as standard ORS in adults with cholera. However, it is sometime associated with an increased incidence of transient, asymptomatic hyponatraemia.

Hydration is one of the important adjuvant therapies for recovery from illness. Euhydrated people have faster recovery rates compared to dehydrated patients from the similar ailments. Hydration can be active (for example, through sodium glucose pump activation, faster hydration rate) or passive (for example, through passive absorption of water, relatively slower hydration rate).

Active hydration can be achieved via sodium-glucose pump, where the usual sodium recommendation for such application is in the range of 60 to 90 mEq/1. This sodium recommendation, however, may not be suitable for elderly or chronic consumption.

Similarly, people suffering with compromised immunity (e.g., geriatric patients) and/or recurring infections are often in need of hydration, preferably active hydration, and multi minerals, vitamins known to support immune functions (e.g. Zinc, Selenium, ascorbic acid).

Diabetes is a recognized risk factor for dehydration. These increased risks of dehydration in diabetes suffering individuals are due to increase glucose secretion in urine, osmotic diuresis caused increased urination, use of anti-hyperglycemic drugs, insufficient fluid intake. Dehydration in diabetes suffering individuals may cause dizziness, headache, tiredness, dry eyes, dry mouth, increased thirst, dark yellow urine, diabetic ketoacidosis, confusion, low blood pressure and weak pulse, hyperosmolar hyperglycemic state. Adequate hydration may help prevent these associated complications.

Further, patients may experience taste fatigue due to pill burden (main therapy dose), and the addition of another pill and water administration becomes unpleasurable experience.

Finally, there is an increasing trend among consumers to proactively maintain a healthy lifestyle to improve their immunity and energy levels. These goals are often achieved though proper hydration and food supplementation with minerals and vitamins.

It is desirable to provide patients with an electrolyte solution that could foster rehydration while providing immune system supporting ingredients. It is also desirable that said electrolyte solution does not interfere with preexisting medical conditions related to salts or sugars metabolism such as blood pressure or diabetes. It is also desirable that said electrolyte solution could be consumed on a daily basis to proactively boost immune system.

Therefore, a formula with a low sodium and dextrose content is proposed. Further, the product may offer additional benefits from other multiminerals, e.g., zinc, selenium, and vitamin e.g., ascorbic acid and/or Pyridoxin HCl (B6), folic acid (B9) and cyanocobalamin (B12).

### SUMMARY

One aspect of the invention pertains to a ready to drink electrolyte solution comprising: Sodium from 40 to 120 mg/100 ml, Potassium from 50 to 100 mg/100 ml, Chloride from 55 to 90 mg/100 ml, Dextrose from 200 to 600 mg/100 ml and wherein an osmolality of the solution is lower than or equal to 268 mOsm/kg.

This aspect may be combined with a variety of embodiments, in any combination. Thus, in some embodiments, the ready to drink electrolyte solution may further comprise vitamins selected from the group of vitamin B6, vitamin B9, vitamin B12, or combinations thereof. In some embodiments the solution further comprises vitamin C. In some embodiments the solution further comprises vitamin D. In some embodiments the solution further comprises Zinc. In some embodiments the solution further comprises Selenium. In some embodiments the solution further comprises Citrate anions. In some embodiments the solution further comprises an antioxidant and/or a chelating agent. In some embodiments the solution further comprises a chelating agent, an antioxidant and at least one vitamin, where the chelating agent and the antioxidant prevent degradation of the at least one vitamin. In some embodiments the solution further comprises a sweetening agent. In some embodiments the solution is substantially free of protein. In one or more embodiments the solution has an energy content less than 10 Kcal/100 ml.

Another aspect of the invention pertains to a method for the manufacture of a ready to drink electrolyte solution, comprising the steps of: Mixing salts, acids, sugars and optional sweeteners in water to create a mixture; Adding vitamins to the mixture; and Diluting the mixture with purified water to obtain a ready to drink electrolyte solution.

This aspect may be combined with a variety of embodiments, in any combination. Thus, in some embodiments, a dissolved oxygen level is controlled to remain under 20 ppm during manufacture. In some embodiments the method comprises a further step of packaging, wherein the ready to drink electrolyte solution is conditioned in a sealed packaged after deaeration and nitrogen purge.

Another aspect of the invention pertains to the use of a ready to drink electrolyte solution as disclosed above, for the rehydration of a human. The invention also pertains to the use of a ready to drink electrolyte solution as disclosed above, for the rehydration of a human suffering from diabetes.

### DETAILED DESCRIPTION

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

As used herein, the terms “about” or “approximately” mean within an acceptable error range for the particular value as determined by one of ordinary skill in the art, which will depend in part on how the value is measured or determined (i.e., the limitations of the measurement system). For example, “about” can mean within three or more standard deviations per practice in the art. Alternatively, “about” can mean a range of up to 10%, up to 5%, up to 3%, up to 1%, or up to 0.5% of a given value. Particularly with respect to systems or processes, the term can mean within an order of magnitude, for example, within five-fold or within two-fold of a value.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

As used herein, the terms “/100 ml” or “per 100 ml” refers to the amount, expressed in weight, of an ingredient(s) present in a 100 ml of the ready to drink electrolyte solution.

As used herein, when a range of values is provided, the measurement unit of the range is similar for both the minimal value and the maximal value, unless otherwise specified. For example, the range “1 to 100 mg” means from “1 mg to 100 mg”.

The ready to drink electrolyte solution of the present disclosure may advantageously provide a fluid with a low osmolality, a low content in sodium and dextrose suitable for daily consumption by human being in need of a palatable solution to restore their hydration level.

Advantageously the ready to drink electrolyte solution of the present disclosure will not interfere with preexisting medical conditions such as diabetes, or hypertension, or chronic infections. Advantageously the ready to drink electrolyte solution of the present disclosure will help the immune system of the human being by providing vitamins and minerals known to support the immune functions. The ready to drink electrolyte solution of the present disclosure is also suitable for daily consumption to proactively boost the immune system. The ready to drink electrolyte solution of the present disclosure may be particularly adapted to elderly people.

One aspect of the invention pertains to a ready to drink electrolyte solution comprising:
Sodium from 40 to 120 mg/100 ml
Potassium from 50 to 100 mg/100 ml
Chloride from 55 to 90 mg/100 ml, and
Dextrose from 200 to 600 mg/100 ml,
   wherein an osmolality of the solution is lower than or equal to 268 mOsm/kg.

Such an electrolyte solution is suitable for everyday use without inducing imbalance in ion or glucose metabolism. This electrolyte solution could participate in an active rehydration of a human. This electrolyte solution could be well balanced for humans suffering from high blood pressure and those in need of rehydration. Finally, this electrolyte solution could be well balanced for human suffering from diabetes and in need of rehydration.

By “ready to drink” it is meant that the solution is designed to be drank or ingested as such, without any further dilution or addition of any solid or liquid.

Preferably, the amount of Sodium in the ready to drink electrolyte solution should remain under 100 mg/100 ml, or under 80 mg/100 ml, or under 60 mg/100 ml. 55 mg/100 ml. For example, the Sodium content of the ready to drink electrolyte solution may range from 40 to 100 mg/100 ml, or 50 to 100 mg/100 ml, or 40 to 80 mg/100 ml, or 50 to 80 mg/100 ml, or 40 to 60 mg/100 ml, or 45 to 55 mg/100 ml, or 45 to 50 mg/100 ml, or 50 to 55 mg/100 ml.

Preferably, the amount of Potassium in the ready to drink electrolyte solution should remain under 90 mg/100 ml, or under 80 mg/100 ml. For example, the Potassium content of the ready to drink electrolyte solution may range from 50 to 90 mg/100 ml, or 50 to 80 mg/100 ml, or 50 to 70 mg/100 ml, or 60 to 100 mg/100 ml or 60 to 90 mg/100 ml, or 60 to 80 mg/100 ml.

It is preferred to have low contents of sodium and/or potassium to provide a ready to drink electrolyte solution suitable for people suffering from metabolism diseases related to sodium or potassium salts, or to be suitable for people willing to control their sodium or potassium salts intake as part of a daily routine. However, a minimal value of sodium and/or potassium is required to assist in the active hydration of the body, via sodium-glucose pump for example.

Preferably, the amount of Chloride in the ready to drink electrolyte solution should remain under 85 mg/100 ml, or under 80 mg/100 ml. For example, the Chloride content of the ready to drink electrolyte solution may range from 55 to 85 mg/100 ml, or 60 to 85 mg/100 ml, or 65 to 85 mg/100 ml, or 70 to 85 mg/100 ml, or 60 to 80 mg/100 ml, or 65 to 80 mg/100 ml, or 70 to 80 mg/100 ml.

Preferably, the amount of dextrose in the ready to drink electrolyte solution should remain under 550 mg/100 ml, or under 500 mg/100 ml. For example, the dextrose content of the ready to drink electrolyte solution may range from 200 to 550 mg/100 ml, or 300 to 550 mg/100 ml, or 400 to 550 mg/100 ml, or 450 to 550 mg/100 ml, or 200 to 500 mg/100 ml, or 300 to 500 mg/100 ml, or 400 to 500 mg/100 ml, or 300 to 550 mg/100 ml, or 300 to 500 mg/100 ml, or 300 to 500 mg/100 ml, or 350 to 500 mg/100 ml.

By “dextrose” it is meant anhydrous dextrose, not dextrose monohydrate.

It is preferred to have a content of dextrose below 550 mg/100 ml to provide a ready to drink electrolyte solution suitable for people suffering from diabetes or other glucose metabolism diseases, or to be suitable for people willing to control their carbohydrate intake as part of a daily routine. However, a minimal value of dextrose is required to assist in the active hydration of the body via sodium-glucose pump for example.

The molar ratio between dextrose and sodium is preferably from 2:1 to 1:2, preferably at about 1:1.

Preferably the osmolality of the ready to drink electrolyte solution should be above 50 mOms/kg, or above 100 mOms/kg, or above 150 mOms/kg, or above 200 mOms/kg.

Preferably the osmolality of the ready to drink electrolyte solution should be about 210 to about 268 mOsm/kg, which corresponds to the WHO criteria for a reduced osmolality solution. However, it is important to note that the ready to drink electrolyte solution according to the present invention is not an oral rehydration solution as defined by the WHO.

The ready to drink electrolyte solution according to the present invention may further comprise vitamins selected from the group of vitamin B6, vitamin B9, vitamin B12, or combinations thereof.

Vitamins of the B group are known to provide many benefits. Vitamin B6 is known to be a key regulator of cell growth and to help restore cell-mediated immunity. Vitamin B6 is known to have anti-inflammatory properties and play a crucial role in the immune response. Also, vitamin B6 supplementation may increase the number/percentage of T-lymphocyte cells, T-helper cells, and T-suppressor cells in critically ill patients. Vitamin B9 can increase innate immunity in elderly people. Vitamin B9 is also known to alter age-associated decrease in NK-cell activity. Vitamin B9 may also support Th1 response. Vitamin B12 is known to increase the numbers of cells with a role in cell-mediated immunity. Adequate dietary level of Vitamin B9 and B12 can act as preventative measures for inflammation, immune dysfunction, and disease progression.

The content in vitamins selected in the group of vitamin B6, vitamin B9, vitamin B12 may range from 0.5 to 3 mg/100 ml, or 0.5 to 2.5 mg/100 ml, or 0.5 to 2 mg/100 ml, or 1 to 3 mg/100 ml, or 1 to 2.5 mg/100 ml, or 1 to 2 mg/100 ml. The content in vitamin B6 (e.g., Pyridoxine HCl) may preferably range from 0.8 to 1.4 mg/100 ml, or from 0.9 to 1.3 mg/100 ml, or from 1 to 1.2 mg/100 ml. The content in vitamin B9 (e.g., Folic acid) may preferably range from 0.1 to 0.3 mg/100 ml, or from 0.15 to 0.25 mg/100 ml, or from 0.18 to 0.2 mg/100 ml. The content in vitamin B12 (e.g., Cyanocobalamin) may preferably range from 0.4 to 2 μg/100 ml, or from 0.5 to 1.7 μg/100 ml, or from 0.6 to 1.4 μg/100 ml. Note that μg can also be indicated as “mcg”, it stands for microgram (1 μg=10⁻⁶ g)

The ready to drink electrolyte solution according to the invention may further comprise vitamin C.

The content of vitamin C may range from 2 to 70 mg/100 ml, or 2 to 60 mg/100 ml, or 2 to 50 mg/100 ml, or 2 to 40 mg/100 ml, or 10 to 70 mg/100 ml, or 20 to 70 mg/100 ml, or 30 to 70 mg/100 ml, or 30 to 60 mg/100 ml, or 40 to 50 mg/100 ml.

It is desirable to have a ready to drink electrolyte solution containing vitamin C (e.g., ascorbic acid) since this vitamin is known to help foster immune system.

The ready to drink electrolyte solution according to the invention may further comprise vitamin D, for example selected from the group of vitamin D2, vitamin D3, or combination thereof.

The content in vitamin D in the ready to drink electrolyte solution may range from 1 μg to 50 mg/100 ml, or 1 μg to 10 mg/100 ml, or 0.1 to 50 mg/100 ml, or 0.1 to 10 mg/100 ml, or 1 to 10 mg/100 ml, or 5 to 10 mg/100 ml, or 6 to 8 mg/100 ml.

It is desirable to have a ready to drink electrolyte solution containing vitamin D2 and/or D3 since these vitamins are known to play a role in the metabolism of Calcium and Phosphate, promote healthy bone structure, have an effect on immune functions and reduce inflammation.

The ready to drink electrolyte solution according to the invention may further comprise Zinc.

The content of Zinc in the ready to drink electrolyte solution may range from 0.1 to 20 mg/100 ml, or 0.5 to 15 mg/100 ml, or 1 to 10 mg/100 ml, or 2 to 10 mg/100 ml, or 2 to 5 mg/100 ml, or 1 to 2 mg/100 ml.

It is desirable to have a ready to drink electrolyte solution containing Zinc since this element is known to help foster immune system. Zinc is known to have beneficial effects in intestinal immune functions, to increase cytotoxicity of NK cells, to restore thymulin activity, to increase numbers of cytotoxic T cells, to reduce the number of activated T helper cells (which can contribute to autoimmunity), and to positively impact the immune system of the aging population and consequently prevent or lower the risk of age-related diseases.

The ready to drink electrolyte solution according to the invention may further comprise Selenium.

The content of Selenium in the ready to drink electrolyte solution may range from 1 to 100 μg/100 ml, or 1 to 50 μg/100 ml, or 1 to 20 μg/100 ml; or 2 to 20 μg/100 ml, or 1 to 10 μg/100 ml, or 2 to 10 μg/100 ml, or 1 to 5 μg/100 ml, or 2 to 5 μg/100 ml, or 2 to 10 μg/100 ml, or 2 to 6 μg/100 ml.

It is desirable to have a ready to drink electrolyte solution containing Selenium since this element is known to help foster the immune system. Selenium is known to have an Antioxidant role, to improve cell-mediated immunity, to improve T helper cell counts, and to enhance immune response to bacterial and viral infections.

The ready to drink electrolyte solution according to the invention may further comprise Citrate anions.

The content of Citrate anions in the ready to drink electrolyte solution may range from 0.1 to 1 g/100 ml, or 100 to 700 mg/100 ml, or 100 to 500 mg/100 ml, or 200 mg to 1 g/100 ml, or 200 mg to 700 mg/100 ml, 200 mg to 500 mg/100 ml, or 300 mg to 700 mg/100 ml, or 300 mg to 500 mg/100 ml, or 150 to 400 mg/100 ml.

The ready to drink electrolyte solution to the invention may further comprise an antioxidant and/or a chelating agent.

The antioxidant may be selected from Vitamin E or derivatives of Vitamin E such as D-α-tocopheryl polyethylene glycol succinate (Vitamin E TPGS), Lecitin, gallic acid and its derivatives, tartaric acid, glucose oxidase, 4-hexyl resorcinaol. Anoxomer, dilauryl thiodipropionate, distearyl thiodipropionate, guaiac resin, isopropyl citrate, butyrated hydroxy anisole. Preferably the antioxidant is a derivative of Vitamin E such as D-α-tocopheryl polyethylene glycol succinate (Vitamin E TPGS). Vitamin E TPGS also provides a source of Vitamin E since Vitamin E accounts for about 25% of the total mass of Vitamin E TPGS.

Preferably the amount of antioxidant in the ready to drink electrolyte solution is from 1 to 50 mg/100 ml, or 2 to 30 mg/100 ml; or 2 to 20 mg/100 ml, or 5 to 20 mg/100 ml, or 10 to 20 mg/100 ml.

The chelating agent may be selected from EDTA, Trisodium citrate, Trisodium orthophosphate, Sodium acetate, Sodium gluconate, Sodium dihydrogen citrate, Diacetyltartaric and fatty acid esters of glycerol, Potassium dihydrogen citrate, Potassium gluconate, Tripotassium citrate.

A preferred chelating agent is EDTA.

Preferably the amount of chelating agent is from 1 to 100 mg/100 ml, or 5 to 50 mg/100 ml, or 10 to 50 mg/100 ml, or 10 to 30 mg/100 ml.

It has been observed that the presence of both an antioxidant and a chelating agent is particularly beneficial for the stability of the vitamin C that may be present in the electrolyte solution.

EDTA has also be reported to help improve absorption of Zinc in children and adults.

The ready to drink electrolyte solution according to the invention may further comprise a chelating agent, an antioxidant and at least one vitamin, wherein the chelating agent and the antioxidant prevent degradation of the at least one vitamin.

The ready to drink electrolyte solution according to the invention may further comprise a sweetening agent.

For clarity, dextrose already present in the electrolyte solution has a sweetening effect, so the solution may comprise another sweetener (or sweetening agent) in addition to dextrose.

The sweetening agent may be selected among natural or artificial sweeteners. A natural sweetener may be selected among: Steviol glucosides (such as Stevia), monk fruit, polyols or sugar alcohols, Allulose, Glycyrrhizin, Thaumatin. An artificial sweetener may be selected among: saccharin, sodium saccharin, aspartame, acesulfame K, neotame, thaumatin, glycyrrhizin, sucralose, cyclamate, dihydrochalcone, alitame, miraculin and monellin and mixtures thereof. Preferably the sweetening agent may be Steviol glucosides, such as Stevia. Preferably the ready to drink electrolyte solution according to the invention may comprise a sweetening agent that does not impact osmolality, such as Stevia.

The content in sweetening agent in the drink electrolyte solution should remain under 100 mg/100 ml, or under 80 mg/100 ml. For example, the sweetening agent content of the ready to drink electrolyte solution may range from 1 to 100 mg/100 ml, or 10 to 100 mg/100 ml, or 20 to 100 mg/100 ml, 40 to 100 mg/100 ml, or 10 to 80 mg/100 ml, or 20 to 80 mg/100 ml, or 40 to 80 mg/100 ml, or 50 to 80 mg/100 ml, or 50 to 70 mg/100 ml.

The ready to drink electrolyte solution according to the invention may be substantially free of protein.

By substantially free of protein, it is meant that the electrolyte solution may contain less than 10 mg/100 ml, or less than 1 mg/100 ml, or less than 0.1 mg/100 ml, of protein.

The ready to drink electrolyte solution according to the invention may have an energy content less than 10 Kcal/100 ml.

The energy content of the ready to drink electrolyte solution should remain under 8 Kcal/100 ml, or under 6 Kcal/100 ml, or under 5 Kcal/100 ml.

Another aspect of the invention pertains to a method for the manufacture of a ready to drink electrolyte solution, comprising the steps of:
Mixing salts, acids, sugars and optional sweeteners in water to create a mixture;
Adding vitamins to the mixture; and
Diluting the mixture with purified water to obtain a ready to drink electrolyte solution.

The method for the manufacture of a ready to drink electrolyte solution may require control of light conditions while performing the addition of vitamins solutions to the mixture. By “controlled light condition” it is meant that the light intensity is reduced compared to normal day light to avoid photochemical degradation of the vitamin. It could be working in a low light environment or working with a modified light spectrum to avoid interaction of a certain wavelength with the vitamin structure.

The method for the manufacture of a ready to drink electrolyte solution according to the invention may require a controlled level of dissolved oxygen in the electrolyte solution to remain under 20 ppm during manufacture.

Preferably the level of dissolved oxygen in the electrolyte solution should remain under 15 ppm, or under 10 ppm, or under 8 ppm, or under 5 ppm, or under 3 ppm, or under 1 ppm during manufacture.

For clarity, 15 ppm is equivalent to 15 mg of Oxygen per 1 liter of electrolyte solution.

It is desirable to keep the level of Oxygen dissolved in the electrolyte solution low to prevent oxidation reaction of the reactive compound, such as the vitamins.

The method for the manufacture of a ready to drink electrolyte solution according to the invention may comprise a further step of packaging, wherein the ready to drink electrolyte solution is conditioned in a sealed packaged after deaeration and nitrogen purge.

Deaeration and nitrogen purge are desirable to remove as much Oxygen as possible from the packaged electrolyte solution, to prevent oxidation reaction of the reactive compound, such as the vitamins.

Another aspect of the invention pertains to the use of a ready to drink electrolyte solution according to the invention for the rehydration of a human.

In other words, the ready to drink electrolyte solution according to the invention may be used for the treatment of dehydration of a human.

Said human in need of rehydration may be of any age superior to 2 years old. Preferably the said human may be of any age superior to 50 years old, or 60 years old, or 70 years old.

Age groups above 50, 60 or 70 years old are more likely to suffer from health issues such as diabetes and/or hypertension; and a ready to drink electrolyte solution with a low sugar and sodium content would be particularly desirable to help them restore their hydration level.

The invention pertains to the use of a ready to drink electrolyte solution according to the invention for the rehydration of a human suffering from diabetes.

In other words, the ready to drink electrolyte solution according to the invention may be used for the treatment of dehydration of a human suffering from diabetes.

Said human being may be suffering from other conditions such as hypertension, or recurring infections for example. Said human being may also be trying to proactively manage its immune system to prevent any infection or medical condition.

### Examples

The following Examples are intended to illustrate, but not to limit, the disclosed subject matter in any manner, shape, or form, either explicitly or implicitly.

### 1—FORMULATION of a Ready to Drink Electrolyte Solution

Table 1 presents comparative examples 1 and 2 which are two electrolyte drinks sold by Johnson & Johnson.

Tables 1 and 2 present inventive examples 3, 4 and 5, which are examples of formulation of a ready to drink electrolyte solution according to the present invention.

TABLE 1

| Ingredients | Comparative | Comparative | Inventive |
|---|---|---|---|
| (g/100 ml) | Example 1 | Example 2 | Example 3 |
| Sucrose | 8.800 | — | — |
| Dextrose Monohydrate | 2.700 | 1.000 | 0.480 |
| Ascorbic Acid | 0.076 | 0.076 | 0.048 |
| DL Malic acid | — | 0.140 | — |
| Sodium Chloride | 0.125 | 0.180 | — |
| Trisodium Citrate Dihydrate | 0.290 | 0.290 | 0.240 |
| Potassium Chloride | 0.150 | 0.150 | 0.150 |
| Concentrated fruit juice (Min 68° Brix) | 1.420 | 1.500 | — |
| Stevia | — | 0.022 | 0.03 |
| Citric acid monohydrate | 0.220 | — | 0.022 |
| Zinc Gluconate | — | — | 0.012 |
| Sodium selenate | — | — | 0.0000072 |
| Vitamins (B6, B9 and B12) | — | — | 0.0013 |
| Disodium edetate | — | — | 0.025 |
| Vitamin E TPGS | — | — | 0.007 |
| Flavoring agent & Colorant | 0.385 | 0.06 | 0.074 |
| Purified water | q.s. 100 ml | q.s. 100 ml | q.s. 100 ml |

TABLE 2

| Ingredients | Inventive | Inventive |
|---|---|---|
| (g/100 ml) | Example 4 | Example 5 |
| Purified water | q.s. 100 ml | q.s. 100 ml |
| Frozen Orange Juice Concentrate | — | 1.636 |
| Dextrose Monohydrate | 0.48 | 0.48 |
| Tri Sodium Citrate Dihydrate | 0.22 | 0.22 |
| Citric Acid Monohydrate | 0.22 | 0.16 |
| Potassium Chloride | 0.15 | 0.15 |
| Ascorbic Acid | 0.04 | 0.04 |
| Stevia (Sigma T5) | 0.03 | 0.025 |
| Disodium Edetate | 0.025 | 0.025 |
| Zinc Gluconate | 0.0115 | 0.0115 |
| Vitamin E - TPGS | 0.007 | 0.007 |
| Flavoring agent & Colorant | 0.075 | 0.039 |
| Sodium Selenate | 0.000014 | 0.000014 |

Example 5 Orange Juice concentrate has a Brix value between 64′ and 660 (Refractometric at 20° C.), an acidity content between 3 and 7 (as % of citric acid anhydrous), and a pH between 3 to 4.3. Nutritional values for 100 g of Orange Juice concentrate are: 271.1 kcal, 2.3 g protein, 61.1 g carbohydrates (including 54.4 g sugars), 0.3 g fat, 2 g fibers, and 32.8 mg salt.

### 2—Manufacturing Procedure

$$Total batch size = 800 liters$$

Step 1: Ingredients were individually added to 50% batch quantity of purified water (except the vitamins and vitamin E TPGS), and optionally fruit juice concentrate.

Step 2: The Vitamin E TPGS was dissolved using hot purified water (approximately 80° C.), once uniform solution observed, the solution is allowed to cool to ambient condition (about 25° C., Room Humidity 60%±10%).

Step 3: Step 2 was added to step 1 and contact time of 1 hour was allowed.

Step 4: The vitamin solutions were added to the solution obtained in step 3 under minimal light and at ambient conditions.

Step 5: The solution obtained in step 4 is adjusted to the target volume with an adequate quantity of purified water.

Step 6: The bulk resulting from Step 5 was divided into 4 different lots namely: A, B, C, D; with different packaging conditions for each lot (see below)
A=with deaeration without nitrogen purge (200 ml per pack).
B=with deaeration with nitrogen purge (180 ml per pack).
C=without deaeration without nitrogen purge (200 ml per pack).
D=without deaeration with nitrogen purge (180 ml per pack).

The product fill volume for packs with nitrogen purge was 180 ml, instead of 200 ml.

“Vitamin E TPGS” stands for D-α-tocopheryl polyethylene glycol succinate.

“Disodium edetate” stands for Disodium ethylenediaminetetraacetate (or EDTA).

### 3—Stabilty and Analysis

Four batches of product were produced and packaged according to the methods disclosed above and analyzed for stability; results are presented in table 3 below.

Example 6 was packaged with deaeration and without nitrogen purge (A).

Example 7 was packaged with deaeration and with nitrogen purge (B).

Example 8 was packaged without deaeration and without nitrogen purge (C).

Example 9 was packaged without deaeration and with nitrogen purge (D).

All four examples 6 to 9 are inventive according to the present invention.

TABLE 3

| | Example 6 | Example 7 | Example 8 | Example 9 |
|---|---|---|---|---|
| Dextrose (g/100 ml) | 0.39 | 0.38 | 0.40 | 0.37 |
| Total carbohydrates (g/100 ml) | 1.12 | 1.05 | 1.15 | 1.06 |
| Fat (g/100 ml) | 0.07 | 0.08 | 0.04 | 0.05 |
| Protein (g/100 mL) | 0.0 | 0.0 | 0.0 | 0.0 |
| Energy (Kcal/100 ml) | 5.05 | 4.86 | 4.94 | 4.67 |
| Stevia (mg/kg) | 59.66 | 62.18 | 58.52 | 61.83 |
| Sodium (mg/100 ml) | 47.92 | 53.75 | 44.35 | 50.06 |
| Potassium (mg/100 ml) | 57.61 | 64.84 | 53.37 | 60.06 |
| Chloride (mg/100 ml) | 74.61 | 85.64 | 70.86 | 78.17 |
| Zinc (mg/100 ml) | 1.292 | 1.48 | 1.08 | 1.332 |
| Selenium (μg/100 ml) | 3.61 | 3.65 | 3.7 | 3.61 |
| Vitamin B6 | 1.10 | 1.12 | 1.11 | 1.16 |
| Pyridoxine HCL (mg/100 ml) | | | | |
| Vitamin B12 (μg/100 ml) | 0.66 | 0.692 | 0.150 | 0.6 |
| (Cyanocobalamin) | | | | |
| Vitamin B9(mg/100 ml) | 0.1929 | 0.1915 | 0.1909 | 0.1927 |
| (Folic acid) | | | | |
| Vitamin C (ascorbic acid) | 45.14 | 44.93 | 39.74 | 39.59 |

Example 7 (with deaeration and with nitrogen purge) was the preferred option; it allows for a better stability of the vitamins present in the formula.

Dextrose, Stevia, Citrate and Vitamin B6 contents were determined by HPLC.
Sodium, Potassium, Zinc and Selenium contents were determined by ICP OES (Inductively coupled plasma—optical emission spectrometry).
Carbohydrate contents were determined by difference, according to AOAC 986.25. “AOAC” stands for Association of Official Agricultural Chemists.
Fat contents were determined according to AOAC 986.25.
Protein contents were determined according to AOAC 920.15.
Vitamin B12 contents were determined by LC MS/MS.
Vitamin B9 contents were determined by ELISA.
Chloride and Vitamin C contents were determined according to FSSAI lab manual.
Energy Kcal contents were determined according to Nutritive value of Indian food (ICMR).

What is claimed is:

1. A ready to drink electrolyte solution comprising:
Sodium from 40 to 120 mg/100 ml;
Potassium from 50 to 100 mg/100 ml;
Chloride from 55 to 90 mg/100 ml; and
Dextrose from 200 to 600 mg/100 ml,
wherein an osmolality of the solution is lower than or equal to 268 mOsm/kg.

2. The ready to drink electrolyte solution according to claim 1, wherein the solution further comprises Vitamins selected from the group of vitamin B6, vitamin B9, vitamin B12, or combinations thereof.

3. The ready to drink electrolyte solution according to claim 1, wherein the solution further comprises vitamin C.

4. The ready to drink electrolyte solution according to claim 1, wherein the solution further comprises vitamin D.

5. The ready to drink electrolyte solution according to claim 1, wherein the solution further comprises Zinc.

6. The ready to drink electrolyte solution according to claim 1, wherein the solution further comprises Selenium.

7. The ready to drink electrolyte solution according to claim 1, wherein the solution further comprises Citrate anions.

8. The ready to drink electrolyte solution according to claim 1, wherein the solution further comprises an antioxidant and/or a chelating agent.

9. The ready to drink electrolyte solution according to claim 1, wherein the solution further comprises a chelating agent, an antioxidant and at least one vitamin, wherein the chelating agent and the antioxidant prevent degradation of the at least one vitamin.

10. The ready to drink electrolyte solution to claim 1, wherein the solution further comprises a sweetening agent.

11. The ready to drink electrolyte solution according to claim 1, wherein said solution is substantially free of protein.

12. The ready to drink electrolyte solution according to claim 1, wherein said solution has an energy content less than 10 Kcal/100 ml.

13. A method for the manufacture of a ready to drink electrolyte solution, comprising the steps
mixing salts, acids, sugars and optional sweeteners in water to create a mixture;
Adding vitamins to the mixture; and
diluting the mixture with purified water to obtain a ready to drink electrolyte solution.

14. The method according to claim 13, wherein a dissolved oxygen level of the mixture is controlled to remain under 20 ppm during manufacture.

15. The method according to claim 13, comprising a further step of packaging, wherein the ready to drink electrolyte solution is conditioned in a sealed packaged after deaeration and nitrogen purge.

16. A method for treating dehydration in a human comprising rehydrating a human with a ready to drink electrolyte solution according to claim 1.

17. A method for treating dehydration in a human suffering from diabetes comprising rehydrating a human suffering from diabetes with a ready to drink electrolyte solution according to claim 1.
